(19) United States
(12) Patent Application Publication
(10) Pub. No.: US 2026/0048178 A1
(43) Pub. Date: Feb. 19, 2026

(54) BIRTH TISSUE FOR SURGICAL REPAIRS

(71) Applicant: LifeNet Health, Virginia Beach, VA (US)

(72) Inventors: Xiaofei Qin, Virginia Beach, VA (US); Fanwei Meng, Virginia Beach, VA (US); Jingsong Chen, Virginia Beach, VA (US); Tak Lung Chang, Virginia Beach, VA (US); Alexander Huber, Virginia Beach, VA (US)

(21) Appl. No.: 19/100,515

(22) PCT Filed: Aug. 2, 2023
(86) PCT No.: PCT/US2023/029292

§ 371 Date: Jan. 31, 2025

Related U.S. Application Data
(60) Provisional application No. 63/394,435, filed on Aug. 2, 2022.

(51) Int. Cl. A61L 27/36; A61L 27/54

(52) U.S. Cl. CPC A61L 27/3604; A61L 27/3645; A61L 27/54; A61L 2300/30; A61L 2430/24

(57) ABSTRACT

The present invention provides a birth tissue derived implant for treating a subject. The birth tissue derived implant comprises a birth tissue and an agent exogenous to the birth tissue. The birth tissue is isolated from an amniotic sac, an umbilical cord or a placental plate. A method is also provided for repairing a defective tissue in a subject. The repair method comprises delivering the birth tissue derived implant to a surface of the defective tissue in the subject. The defective tissue may be an ocular membrane, a synovium, tendon, ligament, nerve, a cartilage or a bone. A method is further provided for preparing the birth tissue derived implant. The preparation method may comprise devitalizing or decellularizing a birth tissue from an amniotic sac, an umbilical cord or a placental plate, and adding an agent to the devitalized or decellularized birth tissue.

### CROSS-REFERENCE TO RELATED APPLICATION

This application claims priority to U.S. Provisional Application No. 63/394,435, filed Aug. 2, 2022, and the contents of which are incorporated herein by reference in their entireties for all purposes.

### FIELD OF THE INVENTION

The invention relates generally to a birth tissue derived implant and uses thereof in repairing a defective tissue.

### BACKGROUND OF THE INVENTION

Various sources of tissues have been utilized by researchers to develop implants for tissue repair. Birth tissues have a long history of use for treating burns and wounds. Amniotic tissues have been used in an ophthalmic setting to support damaged tissues, protect and shield defects from further degeneration or breakdown for decades. There remains a need for birth tissue derived products with optimized biological factors and extracellular matrix components in a user-friendly format.

### SUMMARY OF THE INVENTION

The present invention relates to a birth tissue derived implant and uses thereof for repair a tissue, and provides multiple options to prepare a birth tissue and maximize the healing potential for various clinical applications including defective tissue repair.

A birth tissue derived implant is provided. The birth tissue derived implant comprises an implant birth tissue and an agent exogenous to the implant birth tissue. The implant birth tissue is isolated from an amniotic sac, an umbilical cord, a placental plate, or a combination thereof.

The agent may comprise a synthetic material. The agent may comprise a first birth tissue elute, first birth tissue particulates, a radioprotectant, a biological factor, an immunomodulatory factor, a homeostasis factor, an anti-inflammatory factor, a protease inhibitor, an antioxidant, or a combination thereof. The first birth tissue elute may comprise a growth factor, cytokine, extracellular matrix molecule, extracellular vesicle, protease inhibitors, or combination thereof.

The implant birth tissue may comprise a second birth tissue elute, a second birth tissue particulates, or a combination thereof.

The birth tissue derived implant may further comprise a third birth tissue elute, third birth tissue particulates, or a combination thereof.

The implant birth tissue may be fenestrated, meshed, multi-layered, or crosslinked. The implant birth tissue may be in the form of particulates. The implant birth tissue may have been decellularized, pre-sutured, freeze-dried, lyopreserved, plasticized, oven-dried, heat treated, cryopreserved or terminally sterilized.

The implant birth tissue may have a shape selected from the group consisting of a circle, ellipse, triangle, trapezium, rhombus, polygon, ring, crescent, semicircle, and wedge. The implant birth tissue may be a round piece having two or more cuts from edge to center of the round piece.

The implant birth tissue may comprise cells exogenous to the implant birth tissue. The implant birth tissue may comprise cells endogenous to the amniotic sac, umbilical cord or placental plate from which the implant birth tissue is isolated.

The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer may be on opposite surfaces of the first umbilical cord layer. The implant birth tissue may further comprise a second umbilical cord layer isolated from the first umbilical cord or a second umbilical cord, the second umbilical cord layer may comprise a second umbilical cord lining from the first umbilical cord or the second umbilical cord and a second Wharton's Jelly layer from the first umbilical cord or the second umbilical cord, and the second umbilical cord lining and the second Wharton's Jelly layer may be on opposite surfaces of the second umbilical cord layer. The first umbilical cord lining may be in contact with the second umbilical cord lining. The first Wharton's Jelly layer may be in contact with the second Wharton's Jelly layer.

The implant birth tissue may comprise a first umbilical cord layer isolated from a first umbilical cord, the first umbilical cord layer may comprise a first Wharton's Jelly layer from the first umbilical cord, the first Wharton's Jelly layer may comprise a first subamniotic Wharton's Jelly layer from the first umbilical cord and a first intervascular Wharton's Jelly layer from the first umbilical cord, and the first subamniotic Wharton's Jelly layer and the first intervascular Wharton's Jelly layer may be on opposite surfaces of the first umbilical cord layer.

The implant birth tissue may comprise a first umbilical cord layer isolated from a first umbilical cord, the first umbilical cord layer may comprise a first Wharton's Jelly layer from the first umbilical cord, the first Wharton's Jelly layer may comprise a first subamniotic Wharton's Jelly layer from the first umbilical cord and a first perivascular Wharton's Jelly layer from the first umbilical cord, and the first subamniotic Wharton's Jelly layer and the first perivascular Wharton's Jelly layer may be on opposite surfaces of the first umbilical cord layer.

The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, and the first amnion layer and the first trophoblast layer may be on opposite surfaces of the first placental membrane. The implant birth tissue may further comprise a second placental membrane isolated from the first amniotic sac or a second amniotic sac, the second placental membrane may comprise a second amnion layer from the first amniotic sac or the second amniotic sac and a second chorion layer from the first 1o amniotic sac or the second amniotic sac, the second chorion layer may comprise a second trophoblast layer from the first amniotic sac or the second amniotic sac, and the second amnion layer and the second trophoblast layer may be on opposite surfaces of the second placental membrane. The first amnion layer may be in contact with the second amnion layer or the second trophoblast layer.

The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, and the first amnion layer and the first trophoblast layer may be on opposite surfaces of the first placental membrane. The implant birth tissue may further comprise a second placental membrane isolated from the first amniotic sac or a second amniotic sac, the second placental membrane may comprise a second chorion layer from the first amniotic sac or the second amniotic sac, the second chorion layer may comprise a second cellular layer from the first amniotic sac or the second amniotic sac and a second trophoblast layer from the first amniotic sac or the second amniotic sac, and the second cellular layer and the second trophoblast layer may be on opposite surfaces of the second placental membrane. The first trophoblast layer may be in contact with the second chorion layer or the second trophoblast layer.

The implant birth tissue may comprise a first placental membrane isolated from a first amniotic sac, the first placental membrane may comprise a first chorion layer from the first amniotic sac, the first chorion layer may comprise a first trophoblast layer from the first amniotic sac and a first chorionic membrane layer from the first amniotic sac, and the first trophoblast layer and the first chorionic membrane layer may be on opposite surfaces of the first placental membrane.

The implant birth tissue may consist of a placental plate. The placental plate may be in the form of a particulates.

A method for repairing a defective tissue in a subject is also provided. The repair method comprises (a) delivering a birth tissue derived implant of the present invention to a defective tissue surface of a defective tissue in a subject; and (b) placing the birth tissue derived implant in contact with the defective tissue surface. The birth tissue derived implant may be delivered with a delivery device. The delivery device may be in a rod shape comprising a core, an inner shield and an outer shield. The birth tissue derived implant may be located between the inner shield and the outer shield. The core may comprise a stapler, glue dispenser, suture device, or combination thereof.

Where the implant birth tissue comprises a placental membrane having a trophoblast layer facing the outer shield and an amnion layer facing the inner shield in the delivery device, the repair method may further comprise: (a) inserting the delivery device to a surgical space where the defective tissue is located; (b) removing the outer shield from the delivery device; and (c) rolling the delivery device after step (b) on the defective tissue surface such that the trophoblast layer is in contact with the defective tissue surface.

Where the implant birth tissue comprises an umbilical cord having a Wharton's Jelly layer facing the outer shield and an umbilical cord lining facing the inner shield in the delivery device, the repair method may further comprise: (a) inserting the delivery device to a surgical space; (b) removing the outer shield from the delivery device; and (c) rolling the delivery device after step (b) on the defective tissue surface such that the Wharton's Jelly layer may be in contact with the defective tissue surface.

Where the defective tissue is a defective cartilage, the repair method may further comprise placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may face the defective tissue surface. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the defective tissue surface. Where a bone site defect is under the defective cartilage, the cartilage repair method may further comprise placing a bone graft into the bone site defect before delivering the birth tissue derived implant.

Where the defective tissue is a defective bone, the bone repair method may further comprise placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may 1o face the defective tissue surface. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the defective tissue surface.

Where the defective tissue is a defective dura that the defect led to an opening between epidural space and spinal cord, the dura repair method may further comprise closing the opening with sutures or grafts and placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the umbilical cord lining may face the epidural space and the Wharton's Jelly layer may face the dura. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the dura and the amnion layer may face the epidural space.

Where the defective tissue is a defect on ligamentum flavum, arachnoid membrane, or other sounding soft tissue after laminectomy, the repair method may further comprise placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may face the defective tissue surface and the umbilical cord lining may face the epidural space toward dura, or the Wharton's Jelly layer may face the dura surface and the umbilical cord lining may face the epidural space toward ligamentum flavum, arachnoid membrane, or other sounding soft tissue. The implant birth tissue may comprise a first placental membrane isolated from a first amniotic sac, the first placental membrane may comprise a first amnion layer from the first amniotic sac and a first chorion layer 1o from the first amniotic sac, the first chorion layer may comprise a first trophoblast layer from the first amniotic sac, the first amnion layer and the first trophoblast layer may be on opposite surfaces of the first placental membrane, and the trophoblast layer may face the defective tissue surface and the amnion layer may face the epidural space toward dura, or the trophoblast layer may face the dura surface and the amnion layer may face the epidural space toward the ligamentum flavum, arachnoid membrane, or other sounding soft tissue.

Where the defective tissue is a defective nerve tissue having a defective nerve site, the repair method may further comprise placing the birth tissue derived implant around the defective nerve site or inserting the birth tissue derived implant in the defective nerve site. The repair method may further comprise delivering the birth tissue within a lumen of a tubular bridging device. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer are on opposite surfaces of the first umbilical cord layer. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, and the first amnion layer and the first trophoblast layer may be on opposite surfaces of the first placental membrane.

Where the implant birth tissue comprises a first umbilical cord layer isolated from a first umbilical cord, the first umbilical cord layer comprises a first umbilical cord lining from the first umbilical cord and a first Wharton's Jelly layer from the first umbilical cord, and the first umbilical cord lining and the first Wharton's Jelly layer are on opposite surfaces of the first umbilical cord layer, a repair method comprises delivering the birth tissue derived implant to a defective tissue surface of a defective tissue in a subject. The subject may suffer from synovitis, bursitis, or osteoarthritis (OA), and the defective tissue may be in a joint having an interior joint cavity and forms a defective interior joint surface. The repair method further comprises placing the birth tissue derived implant into the joint, and suturing the birth tissue derived implant onto the defective interior joint surface such that the first Wharton's Jelly layer faces the defective interior joint surface and the first umbilical cord lining faces the interior cavity. The repair method may further comprise contacting the first Wharton's Jelly layer with the defective tissue. The joint may be a knee, ankle, facet spine, wrist, finger, toe, hip, shoulder or elbow joint.

Where the implant birth tissue comprises a first placental membrane isolated from a first amniotic sac, the first placental membrane comprises a first amnion layer from the first amniotic sac and a first chorion layer from the first amniotic sac, the first chorion layer comprises a first trophoblast layer from the first amniotic sac, and the first amnion layer and the first trophoblast layer are on opposite surfaces of the first placental membrane, a repair method comprises delivering the birth tissue derived implant to a defective tissue surface of a defective tissue in a subject. The subject suffers from synovitis, bursitis, or osteoarthritis (OA), and the defective tissue is in a joint having an interior joint cavity and forms a defective interior joint surface. The repair method further comprises placing the birth tissue derived implant into the joint, and suturing the birth tissue derived implant onto the defective interior joint surface such that the first trophoblast layer faces the defective interior joint surface and the first amnion layer faces towards the interior cavity. The repair method may further comprise contacting the first trophoblast layer with the defective tissue. The joint may be a knee, ankle, facet spine, wrist, finger, toe, hip, shoulder or elbow joint.

The repair method may further comprise fixing the birth tissue derived implant to the defective tissue.

The repair method may further comprise delivering the birth tissue derived implant to the defective tissue in a surgery. Where the birth tissue derived implant is delivered in a non-permeable container to the defective tissue, the repair method may further comprise removing the non-permeable container such that the birth tissue derived implant is contact with the defective tissue.

A method of preparing a birth tissue derived implant is further provided. The preparation method comprises (a) devitalizing and/or decellularizing an implant birth tissue isolated from an amniotic sac, an umbilical cord, a placental plate, or a combination thereof; and (b) adding an agent exogenous to the implant birth tissue to the devitalized and/or decellularized implant birth tissue from step (a). The agent may comprise a synthetic material.

According to the preparation method, the implant birth tissue may comprise an umbilical cord layer isolated from an umbilical cord, and the umbilical cord layer may comprise an umbilical cord lining and a Wharton's Jelly layer on opposite surfaces of the umbilical cord layer.

According to the preparation method, the implant birth tissue may comprise a placental membrane isolated from an amniotic sac, the placental membrane may comprise an amnion layer and a chorion layer, the chorion layer may comprise a trophoblast layer, and the amnion layer and the trophoblast layer may be on opposite surfaces of the placental membrane. The preparation method may further comprise treating the placental membrane with a birth tissue elute. The preparation method may further comprise drying, freeze-drying, plasticizing and/or cryopreserving the placental membrane. The preparation method may further comprise decellularizing the placental membrane without separating the amnion layer from the chorion layer in the placental membrane.

According to the preparation method, the implant birth tissue may consist of a placental plate. The placental plate may be in the form of particulates.

For each preparation method of the present invention, a birth tissue derived implant prepared according to the method is provided.

### BRIEF DESCRIPTION OF THE DRAWINGS

FIGS. 1A-D are diagrams illustrating a cross section of a delivery device in a rod shape (A) including a core, an inner shield (C: core+inner shield), birth tissue sheet (D) and outer shield (C), with the core in the center covered by the inner shield, birth tissue sheet and outer shield in an order from the center to the outside of the delivery device.

FIGS. 2A-E show cut patterns of a fenestrated or meshed birth tissue for ocular surface applications.

FIGS. 3A-B show a tubular bridging device incorporated with a birth tissue for nerve regeneration applications. A. the side view. B shows the cross-sectional view.

FIG. 4 shows anti-inflammatory effects of extractions from a decellularized placental membrane (Decell PM) and an enhanced Decell PM. Extractions from both PM types effectively reduced TNF-alpha secretion from lipopolysaccharide (LPS) stimulated THP1 cells. However, the extractions of lyophilized, enhanced Decell PM led to higher TNF-alpha reduction (55.2% from LPS control) when compared to those of the lyophilized Decell PM (36.9% from LPS control)

FIG. 5 shows MMP1 inhibitory effects of amniotic membrane (AM) and enhanced AM. Extractions of the enhanced AM led to a higher MMP1 inhibition (7.3% from the positive control) when compared to those of the AM (0.5% from the positive control).

FIGS. 6A-B show a decellularized placental membrane was used for synovium on-lay in a cadaveric knee joint: (A) placing the decellularized placental membrane with its trophoblast layer down to the soft tissue, and (B) suturing the decellularized placental membrane to the soft tissue.

### DETAILED DESCRIPTION OF THE INVENTION

The present invention relates to a birth tissue derived implant and preparation and uses thereof for repairing a defective tissue in a subject. The inventors have surprisingly discovered that a birth tissue isolated from an amniotic sac (e.g., a placental membrane), umbilical cord or a placental plate may be incorporated into an 1o implant for repair of a defective tissue (e.g., defective joint or cartilage) in a subject, and placing the trophoblast layer of the placental membrane or the Wharton's Jelly layer of the umbilical cord facing the surface of the defective tissue improves healing, regeneration and remodeling. Further, the invention provides methods for preparing the birth tissue derived implant and methods of delivering for more user-friendly applications.

The term “birth tissue derived implant” used herein refers to an implant comprising a birth tissue. The birth tissue derived implant may comprise an agent exogenous to the birth tissue.

The term “birth tissue” used herein refers to a tissue isolated from an amniotic sac, umbilical cord, placental plate, or a combination thereof, optionally modified after the isolation. The amniotic sac, umbilical cord or placental plate may be native or modified. A native amniotic sac, umbilical cord or placental plate comprises multiple layers of tissues. A modified amniotic sac, umbilical cord or placental plate comprises one or some of the layers from the native amniotic sac, umbilical cord or placental plate, respectively. A birth tissue isolated from a native amniotic sac, umbilical cord, or placental plate may comprise all of the layers from the native amniotic sac, umbilical cord, or placental plate. A birth tissue isolated from a modified amniotic sac, umbilical cord, or placental plate may comprise one or some of the layers from the native amniotic sac, umbilical cord, or placental plate. Two or more birth tissues may be isolated from the same amniotic sac, umbilical cord or placental plate.

The term “native” used herein refers to original of a biological material, for example, organism, microorganism, a tissue or cells that is naturally occurring. A modified biological material may be prepared by modifying a native biological material.

The term “agent” used herein refers to a chemical compound, a biological molecule, or a combination thereof.

The term “endogenous” used herein refers to the source of an agent, for example, a chemical compound, biological molecule, or combination thereof, that exists naturally in a biological material (e.g., tissue).

The term “exogenous” used herein refers to the source of an agent, for example, a chemical compound, biological molecule, or combination thereof, that does not derive from the same biological material (e.g., tissue). An exogenous agent may be introduced into the biological material. An agent exogenous to a birth tissue isolated from an amniotic sac, umbilical cord or placental plate may be obtained from a different birth tissue isolated from the same amniotic sac, umbilical cord or placental plate.

The term “amniotic sac” as used herein refers to a thin but tough membranous tissue that holds amniotic fluid in which an embryo and later a fetus develops. The amniotic sac may be native or modified. A native amniotic sac comprises an inner layer (i.e., an amnion layer) and an outer layer (i.e., a chorion layer). A native amnion layer in an amniotic sac comprises several sub-layers, for example, an epithelium, a basement membrane, a compact layer, a fibroblast layer, and a spongy layer (from inside to outside). Similarly, a native chorion layer in an amniotic sac comprises several sub-layers, for example, a cellular layer, a reticular layer, a pseudo-basement membrane, and a trophoblast layer (from inside to outside). The native amnion layer and the native chorion layer in an amniotic sac each comprise cells as well as cellular and extracellular molecules (e.g., growth factors, enzymes, and extracellular matrix molecules). A modified amniotic sac does not comprise all of the layers and sub-layers, cells or cellular and extracellular molecules of a native amniotic sac. A birth tissue isolated from a native amniotic sac may comprise all of the layers and sub-layers, cells or cellular and extracellular molecules from the native amniotic sac. A birth tissue isolated from a modified amniotic sac may comprise one or some of the layers or sub-layers, cells or cellular or extracellular molecules from a native amniotic sac.

The term “placental membrane” used herein refers to a tissue that is derived from amniotic sac and includes an amnion layer (also known as amniotic membrane), a chorion layer or both from the amniotic sac. The placental membrane may be native or modified. A native placental membrane comprises an amnion layer, a chorion layer, or both from the amniotic sac. The chorion layer from an amniotic sac comprises sub-layers, for example, a cellular layer, reticular layer, pseudo-basement membrane, and a trophoblast layer. A modified placental membrane may not comprise the amnion layer or the chorion layer from an amniotic sac. A modified placental membrane may comprise the amnion layer and the trophoblast layer. A modified placental membrane may comprise the amnion layer, the trophoblast layer, and one or more sub-layers selected from the group consisting of the cellular layer, the reticular layer, the pseudo-basement membrane and a combination thereof. For example, a modified placental membrane may comprise the amnion layer, the trophoblast layer, the cellular layer, the reticular layer, the pseudo-basement membrane, or a combination thereof. A birth tissue isolated from a native placental membrane may comprise all of layers in the native placental membrane from an amniotic sac. A birth tissue isolated from a modified placental membrane may comprise one or some of the layers in a native placental membrane from an amniotic sac. For example, a birth tissue isolated from a modified placental membrane may comprise: the amnion layer and the trophoblast layer; the amnion layer, the trophoblast layer, and one or more sub-layers selected from the group consisting of the cellular layer, the reticular layer, the pseudo-basement membrane and a combination thereof; or the amnion layer, the trophoblast layer, the cellular layer, the reticular layer, the pseudo-basement membrane, or a combination thereof.

The term “umbilical cord” used herein refers to a flexible cordlike structure containing blood vessels and attaching a human or other mammalian fetus to the placenta during gestation. The umbilical cord may be native or modified. A native umbilical cord comprises an amnion layer (also known as an umbilical cord lining), a sub-amniotic Wharton's Jelly layer, an intervascular Wharton's Jelly layer and perivascular Wharton's Jelly layers. A modified umbilical cord may comprise the sub-amniotic Wharton's Jelly layer, the intervascular Wharton's Jelly layer and the perivascular Wharton's Jelly layers. A modified umbilical cord may comprise the amnion layer, the sub-amniotic Wharton's Jelly layer, and the intervascular Wharton's Jelly layer. A birth tissue isolated from a native umbilical cord may comprise all of the layers from the native umbilical cord. A birth tissue isolated from a modified umbilical cord may comprise one or some of the layers from a native umbilical cord.

The term “umbilical cord layer” used herein refers to a sheet form of an umbilical cord that is longitudinally dissected with blood vessels removed. The umbilical cord layer may be native or modified. A native umbilical cord layer comprises an amnion layer (also known as umbilical cord lining) and a Wharton's Jelly layer, while the Wharton's Jelly layer may comprise a sub-amniotic Wharton's Jelly layer, an intervascular Wharton's Jelly layer and perivascular Wharton's Jelly layers. A modified umbilical cord layer may comprise the sub-amniotic Wharton's Jelly layer, the intervascular Wharton's Jelly layer and the perivascular Wharton's Jelly layer. A modified umbilical cord layer may further comprise the amnion layer, the sub-amniotic Wharton's Jelly layer, the intervascular Wharton's Jelly layer. A birth tissue isolated from a native umbilical cord layer may comprise all of the layers from the native umbilical cord layer. A birth tissue isolated from a modified umbilical cord layer may comprise one or some of the layers from a native umbilical cord layer.

The term “placental plate” or “placental disc” used herein interchangeably refers to a mature placenta in a disk like shape, about 1-5 cm thick, and about 15-20 cm in diameter. The maternal side of the placenta plate is dull and is subdivided into as many as 35 lobes. The fetal side of the placental plate may be covered by an amnion, also called an amnion layer or amniotic membrane. The placental plate may be native or modified. The placenta plate may be in the form of particulates. An implant birth tissue consisting of a placental plate does not comprise an amniotic sac, an umbilical cord, an amnion layer or a chorion layer.

The term “birth tissue elute” used herein refers to a supernatant collected from a mixture of birth tissue particulates and a liquid after the mixture has been incubated. A suitable liquid has a neutral pH of, for example, about 6.8-7.8. The liquid may be a culture medium, and the resulting birth tissue elute may also be called a conditioned medium.

Where the birth tissue particulates may be particulates of a birth tissue isolated from an amniotic sac, umbilical cord, placental plate or a combination thereof, after a soluble fraction is removed from the isolated birth tissue to prepare a birth tissue elute, the resulting isolated birth tissue particulates may be used to prepare an implant birth tissue, with or without any further modification, for example, decellularization. The birth tissue elute may be added to the implant birth tissue.

The birth tissue elute may be viscous. The birth tissue elute may have a shear viscosity of 0.1-500, 1-100, 1-50, 0.1-10, 5-45 or 15-45 Pa·s at 1-5 Hz. The shear viscosity of the birth tissue elute may be 5-45 Pa·s at 2.5 Hz or 0.1-10 Pa·s at 0.5 Hz. The birth tissue elute may have a shear viscosity of 0.01-0.2, 0.01-0.15, 0.01-0.1, or 0.05-0.8 Pa·s at strains higher than 10% and a shear viscosity of 0.05-10, 0.05-5, 0.05-2, 0.05-1, or 0.1-1 Pa·s at strains less than 10% at 0.5-1 Hz. The birth tissue elute may be viscous to an extent such that the birth tissue elute may not be flowable in a tube after turning the tube upside down.

The birth tissue elute may be obtained from a non-decellularized birth tissue isolated from, for example, an umbilical cord, a placental membrane, a placental plate, or a combination thereof. The birth tissue elute can be prepared by a method described in US 2022/0152120.

The birth tissue elute may comprise double stranded DNA. The birth tissue elute may have a double stranded DNA of 1-3000, 30-3000, 50-3000, 50-2000, or 100-2000 ng DNA/mL elute.

The birth tissue elute may comprise a variety of solubilized bioactive components. The solubilized bioactive components may include hyaluronic acid (HA), cytokines, growth factors, protease inhibitors, for example, tissue inhibitors of metalloproteinase (TIMPs), and/or chemokines. The birth tissue elute preparation method may further comprise adjusting the concentration of a bioactive component in the elute to a desirable level.

The birth tissue elute may comprise hyaluronic acid (HA). The concentration of the HA in the birth tissue elute may be about 0.01-100, 0.05-50, 0.1-20, 0.5-10 mg, 1-10, or 1-5 mg/mL. The birth tissue elute may not comprise a HA that is not from the birth tissue. The birth tissue elute preparation method may further comprise adjusting the HA concentration in the elute. The HA may contain different molecular weight, from about 5 to about 10,000 kDa, from about 5 to about 8,000 kDa, from about 5 to about 6,000 kDa, or from about 8 to about 6,000 kDa. The HA concentration may be adjusted to a desirable level at, for example, about 4.5-5.5 mg/mL or about 9-11 mg/mL.

The birth tissue elute may comprise one or more cytokines. The cytokine may be interleukin(IL)-1 receptor antagonist (IL-1RA), IL-4, IL-6, IL-8, IL-10, IL-11, and/or IL-13. The concentration of the IL-1RA in the birth tissue elute may be about 10-2000, 50-1000, 50-500, or 10-500 ng/mL. The birth tissue elute may not comprise a cytokine exogenous to the amniotic sac, umbilical cord or placental plate from which the implant birth tissue is isolated. The birth tissue elute preparation method may further comprise adjusting the concentration of the cytokine in the elute. The cytokine concentration may be adjusted to a desirable level at, for example, about 250-350 ng/mL. The birth tissue elute may not comprise IL-1, for example, IL-1beta, at a concentration higher than about 10, 50, 100 or 200 pg/mL.

The birth tissue elute may comprise one or more bioactive factors (e.g., biochemical factors). The bioactive factor may be basic fibroblast growth factor (bFGF or FGF-2), transforming growth factor beta (TGF-beta), platelet derived growth factor—AA (PDGF-AA), platelet derived growth factor—BB (PDGF-BB), transforming growth factor alpha (TGF-alpha), hepatocyte growth factor (HGF), placental growth factor (PIGF), vascular endothelial growth factor (VEGF), growth differentiation factors (GDF), insulin-like growth factor (IGF), insulin-like growth factor binding protein (IGFBP), epidermal growth factor (EGF), stromal cell-derived factor-1 (SDF-1), angiogenin, pentraxin-3 (PTX-3), or granulocyte-colony stimulating factor (GCSF). The concentration of the bFGF in the birth tissue elute may be 1-10,000 ng/mL. The birth tissue elute may not comprise a growth factor exogenous to the amniotic sac, umbilical cord or placental plate from which the implant birth tissue is isolated. The preparation method may further comprise adjusting the concentration of the growth factor in the elute. The growth factor concentration may be adjusted to a desirable level at, for example, about 90-110 ng/mL.

The birth tissue elute may comprise a protease inhibitor. The birth tissue elute may comprise a protease. The birth tissue elute may not comprise a protease inhibitor exogenous to the amniotic sac, umbilical cord or placental plate from which the implant birth tissue is isolated. The protease may be trypsin, serine protease, cysteine protease, threonine protease, aspartic protease, or metalloproteases. The protease inhibitor may be a tissue inhibitor of metalloproteinase (TIMP) and/or alpha-2-macroglobulin (A2M). The TIMP may be of TIMP-1, TIMP-2, TIMP-3, or TIMP-4. The concentration of the TIMP-1 in the birth tissue elute may be about 0.1-100, 0.1-50, 0.1-10, 0.1-5, 0.1-1, 0.1-0.9, 0.1-0.8. 0.1-0.7, 0.1-0.6, 0.1-0.5, 0.1-0.4, 0.1-0.2, 0.5-100, 0.5-50, 0.5-10, 0.5-5, 0.5-1, 1-100, 1-50, 1-30, 1-20 or 1-10 μg/mL. The preparation method may further comprise adjusting the concentration of the TIMP in the elute. The TIMP concentration may be adjusted to a desirable level at, for example, adjust TIMP-1 to about 0.1-0.8, 0.1-0.9, 2.5-3.5 μg/mL or 4.5-5.5 μg/mL. The A2M concentration in the birth tissue elute may be about 0.1-1000, 1-1000, 1-500, 1-100, or 10-100 μg/mL.

The birth tissue elute may comprise extracellular vesicles. The birth tissue elute may not comprise extracellular vesicles exogenous to the amniotic sac, umbilical cord or placental plate from which the implant birth tissue is isolated. The extracellular vesicles may be positive with a biomarker such as CD40+. The number of the extracellular vesicles in the birth tissue elute may be about 10,000-100,000,000, 10,000-50,000,000, 10,000-20,000,000, 1,000,000-100,000,000, 1,000,000-50,000,000, or 1,000,000-20,000,000 per mL. The preparation method may further comprise adjusting the number of the extracellular vesicles in the elute. The extracellular vesicles may be adjusted to a desirable level at, for example, about 10,000,000-50,000,000 per mL or about 50,000,000-100,000,000 per mL.

The birth tissue elute may comprise exosomes. The birth tissue elute may not comprise exosomes exogenous to the amniotic sac, umbilical cord or placental plate from which the implant birth tissue is isolated. The exosomes may be positive with a biomarker such as CD9+. The number of the exosomes in the birth tissue elute may be about 10,000-100,000,000, 10,000-50,000,000, 10,000-20,000,000, 1,000,000-100,000,000, 1,000,000-50,000,000, or 1,000,000-20,000,000 per mL. The preparation method may further comprise adjusting the number of the exosomes in the elute. The exosomes may be adjusted to a desirable level at, for example, about 10,000,000-50,000,000 per mL or about 50,000,000-100,000,000 per mL.

The birth tissue elute may not comprise a substantial amount of solubilized extracellular matrix components. The extracellular matrix components may be selected from the group consisting of collagen, laminin, fibronectin, and a combination thereof. The solubilized extracellular matrix proteins may constitute less than about 0.01, 0.05, 0.1, 0.5, 1, 2, 3, 4, or 5 wt % or mg/ml of the birth tissue elute.

The birth tissue elute may not comprise a substantial amount of solubilized collagen. The solubilized collagen may constitute less than about 0.01, 0.05, 0.1, 0.5, 1, 2, 3, 4, or 5 wt % of the birth tissue elute. The birth tissue elute may comprise less than about 0.01, 0.05, 0.1, 0.5, 1, 2, 3, 4, or 5 mg/ml collagen.

The birth tissue elute may not comprise a substantial amount of solubilized laminin. The solubilized laminin may constitute less than about 5, 3 or 1 wt % or mg/ml of the birth tissue elute. The birth tissue elute may comprise less than about 0.01, 0.05, 0.1, 0.5, 1, 2, 3 or 5 mg/ml laminin.

A bioactive factor in the presence of the birth tissue elute may have a longer shelf-life at different temperatures than the same bioactive factor in the absence of the elute. The elute may extend the shelf-life of the bioactive factor at ambient temperature from about 1 minute to about 48 hours. The elute may extend the shelf-life of the bioactive factor by at least about 10, 100, 500 or 1,000 times. The elute may maintain from about 20% to about 100%, from about 30% to about 100%, from about 30% to about 80%, from about 40% to about 80%, or from about 50% to about 100% of the detectable bioactive factor at ambient temperature for about 24 hours. The elute may maintain from about 20% to about 100%, from about 30% to about 100%, from about 30% to about 80%, from about 40% to about 80%, or from about 50% to about 100% of the detectable bioactive factor at ambient temperature for about 2 days. The elute may maintain the detectable bioactive factor from about 20% to about 100%, from about 30% to about 100%, from about 30% to about 80%, from about 40% to about 80%, or from about 50% to about 100% at about 37° C. for about 24 hours. The elute may maintain from about 20% to about 100%, from about 30% to about 100%, from about 30% to about 80%, from about 40% to about 80%, or from about 50% to about 100% of the detectable bioactive factor at about 37° C. for about 2 days.

The term “particulates” as used herein refers to small pieces, for example, of a birth tissue, also known as birth tissue particulates. The particulates may have an average particle size in the range of about 0.1-10,000, 0.1-5,000, 0.1-2,000, 0.1-1,000, 0.1-500, 0.1-100, 0.1-10, 0.1-1, 0.5-10,000, 0.5-5,000, 0.5-2,000, 0.5-1,000, 0.5-500, 0.5-100, 0.5-10, 0.5-1, 1-10,000, 1-5,000, 1-2,000, 1-1,000, 1-500, 1-100, 1-10, 5-10,000, 5-5,000, 5-2,000, 5-1,000, 5-500, 5-100, 5-10, 10-10,000, 10-5,000, 10-2,000, 10-1,000, 10-500, 10-100, 50-10,000, 50-5,000, 50-2,000, 50-1,000, 50-500, 50-100, 100-10,000, 100-5,000, 100-2,000, 100-1,000 or 100-500 μm. For example, the particulates may have an average particle size in the range of about 10-2,000 μm.

The term “subject” used herein refers to a mammal, for example, human, bovine, porcine, murine, ovine, equine, canine, caprine and feline. The subject may have a defective tissue. For example, the subject is a human having a defective tissue.

The term “suture” used herein refers to a stitch or row of stitches holding together the edges of a wound or surgical incision.

The term “pre-suture” used herein refers to the implant comprise suture in the package as a product.

The term “tubular bridging device” used herein refers to a hollow material used to facilitate nerve regeneration.

The term “mesh” used herein refers to a material having openings between the threads, cords, filaments, fibrils, fibers or fiber bundles.

The term “meshed” used herein refers to openings created to the material, while the openings may be visible as pores after stretch the material.

The term “fenestrated” used herein refers to having perforations in the material or implant, completely or partially in terms of depth of perforation through material or implant.

The term “radioprotectant” used herein refers to an agent that reduces damage to a tissue, for example, a birth tissue in a birth tissue derived implant, caused by radiation.

The term “biological factor” used herein refers to an agent that affects the function or behavior of a living organism, an organ, a tissue, or cells. The biological factor may be an antioxidant, growth factor, cytokine, chemokine, lubricating factor, immunomodulatory factor, homeostasis factor, anti-inflammatory factor, or protease inhibitor.

The terms “antioxidant” and “free radical scavenger” are used herein interchangeably and refer to an agent that suppresses a damage caused by a free radical.

The term “lubricating factor” used herein refers to an agent that minimizes friction and allow smooth movement.

The terms “immunomodulatory factor” and “immunomodulator” are used herein interchangeably and refer to an agent that stimulates or suppresses the immune system in a subject. The immunomodulatory factor or immunomodulator may help the subject fight cancer, infection, or other diseases.

The term “homeostasis factor” used herein refers to a agent that contributes to a process used by a subject to actively maintain fairly stable conditions necessary for survival and health of the subject.

The term “anti-inflammatory factor” used herein refers to an agent that reduces inflammation in a subject, and the inflammation may be measured by the level of an inflammatory factor in the subject.

The term “protease inhibitor” used herein refers to an agent that suppresses the function of a protease.

The term “multi-layered” used herein refers to a process of stacking two or more layers of birth tissue on top of each other.

The term “freeze-dried,” “freeze-drying”, “lyophilized” or “lyophilizing” used herein refers to a process in which water is removed from a sample after the sample is frozen and placed under a vacuum, allowing water to transform directly from solid to vapor without passing through a liquid phase.

The term “plasticized” or “plasticizing” used herein refer to a process of making a sample more flexible.

The term “cryopreserved” or “cryopreserving” used herein refers to a process that preserves a biological sample containing organelles, cells, tissues, or any other biological constructs by cooling the biological sample in a cryopreservation medium to a very low temperature, for example, below the freezing point of the cryopreservation medium.

The term “terminally sterilized” or “terminal sterilization” used herein refers to a process, in which a product is sterilized in its final container or packaging, permitting measurement and evaluation of quantifiable microbial lethality in the product.

The term “a substantial amount” used herein refers to at least about 70%, 80%, 90%, 95%, 99%, 99.9% or 99.99%.

The term “decellularization” or “decellularize” used herein refers to removal of a substantial amount, for example, at least about 70%, 80%, 90%, 95%, 99%, 99.9% or 99.99%, of cells from a tissue, for example, a birth tissue.

The term “devitalization” or “devitalize” used herein refers to killing a substantial amount, for example, at least about 70%, 80%, 90%, 95%, 99%, 99.9% or 99.99%, of viable cells in a tissue, for example, a birth tissue.

The term “pre-sutured” used herein refers to an implant comprising a suture.

The present invention provides a birth tissue derived implant. The birth tissue derived implant may be used for treating a subject. The birth tissue derived implant comprises a birth tissue (also referred to as an implant birth tissue) and an agent exogenous to the implant birth tissue. The implant birth tissue is isolated from an amniotic sac, an umbilical cord, a placental plate, or a combination thereof.

An amniotic sac may be native or modified. An implant birth tissue isolated from an amniotic sac may comprise one, some, or all of the layers from a native amniotic sac.

An umbilical cord or an umbilical cord layer may be native or modified. A native umbilical cord or a native umbilical cord layer comprises an amnion layer. A modified umbilical cord or a native umbilical cord layer may not comprise an amnion layer. An implant birth tissue isolated from an umbilical cord or an umbilical cord layer may comprise one, some or all of the layers from a native umbilical cord or a native umbilical cord layer. For example, an implant birth tissue isolated from an umbilical cord may or may not comprise an amnion layer from a native umbilical cord.

The subject may suffer from a disease or disorder. The subject may suffer from synovitis, bursitis, osteoarthritis (OA), or an ocular surface disease or disorder. The ocular surface disease or disorder may be corneal erosion, corneal ulcer, chemical or thermal burn, neurotrophic ulcer, excessive dry eye, pterygium excision, conjunctival chalasis, fornix reconstruction, symblepharon or Stevens-Johnson syndrome.

The subject may have a tissue defect. The tissue defect may be a cartilage defect, an osteochondral defect, or a nerve defect. The subject may have a cartilage defect and a bone defect under the cartilage defect. The tissue defect may be a synovial defect and/or synovitis.

The subject may be predisposed to a disease or disorder. In one embodiment, the subject is predisposed to fibrosis. The fibrosis may be epidural fibrosis.

The agent may comprise a first birth tissue elute, first birth tissue particulates, a radioprotectant, a biological factor, an immunomodulatory factor, a homeostasis factor, an anti-inflammatory factor, a protease inhibitor, an antioxidant, or a combination thereof.

The agent may comprise a synthetic material. The synthetic material may be resorbable or non-resorbable. The synthetic material may comprise one or more polymers. The synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The agent may be of any form, shape or size.

The synthetic material may be a synthetic mesh. The synthetic mesh may be made of one or more of synthetic materials. The synthetic material may be a synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The mesh may be flat, for example, in the form of a sheet.

The synthetic material may be a synthetic suture. The synthetic suture may be made of one or more synthetic materials. The synthetic material may be a synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The suture may be in an elongated shape, for example, rod or tubular shape, and pass through a material (e.g., tissue) to connect two or more different materials.

The synthetic material may be a synthetic tubular bridging device. The synthetic tubular bridging device may be made of one or more synthetic materials. The synthetic material may be a synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof.

The agent may comprise a first birth tissue elute. The first birth tissue elute may be prepared from a first birth tissue isolated from an amniotic sac, umbilical cord, placental plate, or combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue elute may comprise a growth factor, cytokine, extracellular matrix molecule, protease inhibitors, extracellular vesicle, or combination thereof.

The agent may comprise first birth tissue particulates. The first birth tissue particulates are particulates of a first birth tissue isolated from an amniotic sac, umbilical cord, placental plate, or combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue particulates may comprise a growth factor, cytokine, extracellular matrix molecule, protease inhibitors, extracellular vesicle, or combination thereof.

The agent may comprise a radioprotectant. The radioprotectant may be endogenous or exogenous to the amniotic sac, umbilical cord or placental plate from which the implant birth tissue is isolated. The radioprotectant may be a biological factor. The biological factor may be a recombinant biological factor. The recombinant biological factor may be basic fibroblast growth factor (bFGF).

The biological factor may be an antioxidant, also known as free radical scavenger, for example, ascorbic acid, vitamin E, and polysaccharide.

The biological factor may be a lubricating factor, for example, hyaluronic acid (HA) or lubricin.

The biological factor may be an immunomodulatory factor and homeostasis factor, for example, interleukin-10 (IL-10) and transforming growth factor beta-3 (TGFβ-3).

The biological factor may be an anti-inflammatory factor. The anti-inflammatory factor may be interleukin-1 receptor antagonist (IL-1RA), hepatocyte growth factor (HGF) or pentraxin 3 (PTX3).

The biological factor may be a protease inhibitor. The protease inhibitor may be tissue inhibitor of metalloproteinase 1 (TIMP1), tissue inhibitor of metalloproteinase 2 (TIMP2), tissue inhibitor of metalloproteinase 3 (TIMP3), tissue inhibitor of metalloproteinase 4 (TIMP4), or alpha2 macroglobulin (A2M).

The agent may comprise a birth tissue elute and a radioprotectant. For example, the radioprotectant may be a polysaccharide added to the birth tissue elute before the birth tissue elute is subject to terminal gamma-irradiation for sterilization.

The implant birth tissue may further comprise a second birth tissue elute, a second birth tissue particulates, or a combination thereof.

The second birth tissue elute may be prepared from a second birth tissue isolated from an amniotic sac, umbilical cord, placental plate, or combination thereof. The second birth tissue elute may be prepared from the amniotic sac, umbilical cord, placental plate, or combination thereof from which the implant birth tissue is isolated. The second birth tissue elute may comprise a growth factor, cytokine, extracellular matrix molecule, protease inhibitors, extracellular vesicle, or combination thereof.

The second birth tissue particulates are particulates of a second birth tissue isolated from an amniotic sac, umbilical cord, placental plate, or combination thereof. The second birth tissue particulates may be prepared from the amniotic sac, umbilical cord, placental plate, or combination thereof from which the implant birth tissue is isolated. The second birth tissue particulates may comprise a growth factor, cytokine, extracellular matrix molecule, protease inhibitors, extracellular vesicle, or combination thereof.

The birth tissue derived implant may further comprise a third birth tissue elute. The third birth tissue elute may be prepared from a third birth tissue isolated from an amniotic sac, umbilical cord, placental plate, or combination thereof. The third birth tissue elute may be prepared from the amniotic sac, umbilical cord, placental plate, or combination thereof from which the implant birth tissue is isolated. The third birth tissue elute may comprise a growth factor, cytokine, extracellular matrix molecule, protease inhibitors, extracellular vesicle, or combination thereof.

The birth tissue derived implant may further comprise third birth tissue particulates. The third birth tissue particulates are particulates of a third birth tissue isolated from an amniotic sac, umbilical cord, placental plate, or a combination thereof. The third birth tissue may be isolated from the amniotic sac, umbilical cord, placental plate, or combination thereof from which the implant birth tissue is isolated. The third birth tissue particulates may comprise a growth factor, cytokine, extracellular matrix molecule, protease inhibitors, extracellular vesicle, or combination thereof.

The first elute, the second elute and the third elute are different.

The first particulates, the second particulates and the third particulates are different.

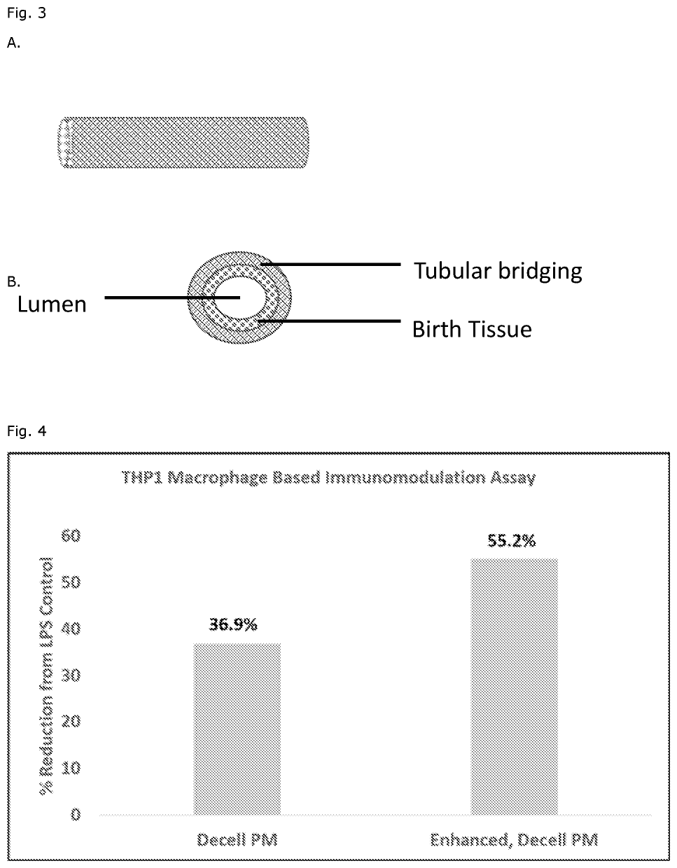

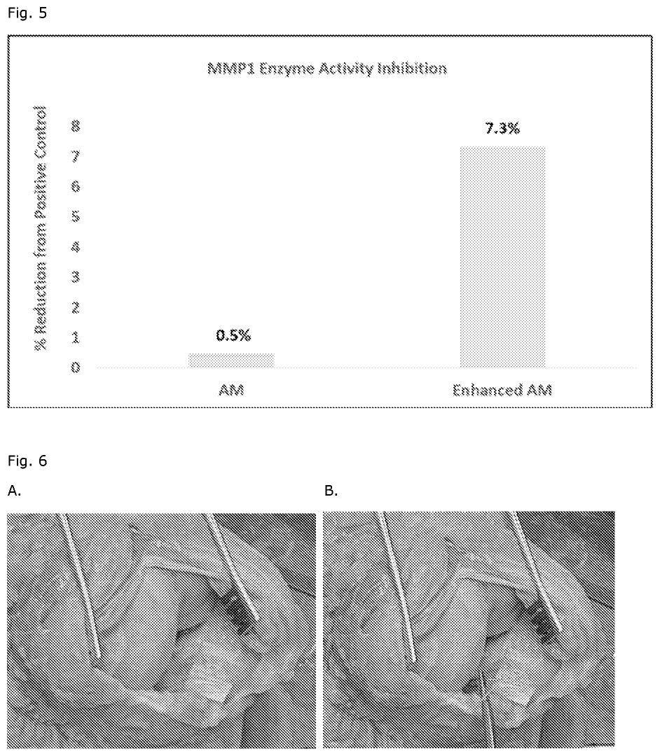

The implant birth tissue may be fenestrated, meshed, multi-layered or crosslinked. The implant birth tissue may implant fenestrated completely or partially. The implant birth tissue may be a round piece having two or more cuts from edge to center of the round piece. FIGS. 3A-E show cut patterns of a fenestrated or meshed implant birth tissue for ocular surface applications. The implant birth tissue may comprise cells. The cells may be viable cells, non-viable cells, or a combination thereof. The cells may be exogenous to the implant birth tissue.

The implant birth tissue may be crosslinked. The extracellular matrix (ECM) in the implant birth tissue may be crosslinked such that hyaluronic acid (HA) in the ECM forms a hyaluronan based hydrogel, which provides fluid absorbency and a stable 3D porous scaffold without denaturing factors. The implant birth tissue may be in the form of a hydrogel. The implant birth tissue may have been freeze-dried, lyopreserved, plasticized, cryopreserved or terminally sterilized.

The implant birth tissue may have been decellularized. A substantial amount, for example, at least about 70, 80, 90, 95, 99, 99.9 or 99.99% of the cells may have been removed from the implant birth tissue.

The implant birth tissue may be fenestrated or meshed in certain patterns.

The implant birth tissue may be pre-sutured.

The implant birth tissue may have any geometric shape, for example, a circle, ellipse, triangle, trapezium, rhombus, polygon, ring, crescent, semicircle, wedge, or combination thereof. The implant birth tissue may form a shape for a defective tissue in need of repair. The shape may match the shape of a defect in a tissue in need of repair.

In one embodiment, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer are on opposite surfaces of the first umbilical cord layer. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface.

The implant birth tissue may further comprise a second umbilical cord layer isolated from the first umbilical cord or a second umbilical cord, the second umbilical cord layer may comprise a second umbilical cord lining from the first umbilical cord or the second umbilical cord, and a second Wharton's Jelly layer from the first umbilical cord or the second umbilical cord such that the second umbilical cord lining and the second Wharton's Jelly layer may be on opposite surfaces of the second umbilical cord layer. The first umbilical cord lining may be in contact with the second umbilical cord lining. The first Wharton's Jelly layer may be in contact with the second Wharton's Jelly layer.

The implant birth tissue may comprise a first umbilical cord layer isolated from a first umbilical cord, the first umbilical cord layer may comprise a first Wharton's Jelly layer from the first umbilical cord, the first Wharton's Jelly layer may comprise a first subamniotic Wharton's Jelly layer from the first umbilical cord and a first intervascular Wharton's Jelly layer from the first umbilical cord, and the first subamniotic Wharton's Jelly layer and the first intervascular Wharton's Jelly layer may be on opposite surfaces of the first umbilical cord layer. The Wharton's Jelly layer may have an uneven surface.

The implant birth tissue may comprise a first umbilical cord layer isolated from a first umbilical cord, the first umbilical cord layer may comprise a first Wharton's Jelly layer from the first umbilical cord, the first Wharton's Jelly layer may comprise a first subamniotic Wharton's Jelly layer from the first umbilical cord and a first perivascular Wharton's Jelly layer from the first umbilical cord, and the first subamniotic Wharton's Jelly layer and the first perivascular Wharton's Jelly layer may be on opposite surfaces of the first umbilical cord layer.

In another embodiment, the implant birth tissue may comprise a first placental membrane isolated from a first amniotic sac. The first placental membrane may comprise a first amnion layer from the first amniotic sac and a first chorion layer from the first amniotic sac. The first chorion layer may comprise a first trophoblast layer from the first amniotic sac. The first amnion layer and the first trophoblast layer may be on opposite surfaces of the first placental membrane.

The implant birth tissue further comprises a second placental membrane isolated from the first amniotic sac or a second amniotic sac. The second placental membrane may comprise a second amnion layer from the first amniotic sac or the second amniotic sac and a second chorion layer from the first amniotic sac or the second amniotic sac. The second chorion layer may comprise a second trophoblast layer from the first amniotic sac or the second amniotic sac, and the second amnion layer and the second trophoblast layer may be on opposite surfaces of the second placental membrane. The first amnion layer may be in contact with the second amnion layer or the second trophoblast layer. The first trophoblast layer may be in contact with the second trophoblast layer.

The implant birth tissue may further comprise a second placental membrane isolated from the first amniotic sac or a second amniotic sac. The second placental membrane may comprise a second chorion layer from the first amniotic sac or the second amniotic sac. The second chorion layer may comprise a second cellular layer from the first amniotic sac or the second amniotic sac and a second trophoblast layer from the first amniotic sac or the second amniotic sac. The second cellular layer and the second trophoblast layer may be on opposite surfaces of the second placental membrane. The first amnion layer may be in contact with the second cellular layer or the second trophoblast layer. The first trophoblast layer may be in contact with the second trophoblast layer or the second cellular layer.

The implant birth tissue may comprise a first placental membrane isolated from a first amniotic sac. The first placental membrane may comprise a first chorion layer from the first amniotic sac. The first chorion layer may comprise a first trophoblast layer from the first amniotic sac and a first chorionic membrane layer from the first amniotic sac. The first trophoblast layer and the first chorionic membrane layer may be on opposite surfaces of the first placental membrane. The first chorionic membrane layer may be in contact with the second cellular layer or the second trophoblast layer. The first trophoblast layer may be in contact with the second trophoblast layer or the second cellular layer.

In another embodiment, the implant birth tissue may comprise a first placental membrane isolated from a first amniotic sac and a second birth tissue isolated from an umbilical cord. The trophoblast layer from the first placental membrane may be in contact with the Wharton's Jelly layer or the umbilical cord lining from the umbilical cord. The amnion layer from the first placental membrane may be in contact with the umbilical cord lining of the umbilical cord layer. The cellular layer from the first placental membrane may be in contact with the Wharton's Jelly layer or the umbilical cord lining from the umbilical cord.

In yet another embodiment, the implant birth tissue may comprise a placental plate. The implant birth tissue may consist of a placental plate. The placenta plate may be in the form of particulates. The implant birth tissue consisting of a placental plate does not comprise an amniotic sac, an umbilical cord, an amnion layer or a chorion layer.

A method for repairing a defective tissue in a subject is provided. The repair method comprises delivering the birth tissue derived implant of the present invention to a surface of a defective tissue (“defective tissue surface”) in a subject. The repair method further comprises placing the birth tissue derived implant in contact with the defective tissue surface. The subject may suffer from synovitis, bursitis, tendonitis, tendinosis, tendinopathy, plantar fasciitis, osteoarthritis (OA), acute gout, chronic gout, back pain, post-surgery tissue adhesion, endometriosis, soft tissue scar, fibrosis, nerve injury, or an ocular surface disease or disorder. The subject may suffer from corneal erosion, corneal ulcer, chemical burn, thermal burn, neurotrophic ulcer, excessive dry eye, pterygium excision, conjunctival chalasis, fornix reconstruction, symblepharon or Stevens-Johnson syndrome. The subject may have had a laparoscopic surgery, cardiothoracid surgery, orthopedic spine surgery, orthopedic neuro-surgery, abdominal surgery, or pelvic surgery. The subject may have a cartilage defect, osteo-chondral defect, or nerve defect. The subject may be predisposed to fibrosis. The fibrosis may be epidural fibrosis.

According to the repair method of the present invention, the birth tissue derived implant may further comprise a synthetic material. The synthetic material may be resorbable or non-resorbable. The synthetic material may comprise one or more synthetic polymers. The synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The agent may be of any form, shape or size.

The synthetic material may be a synthetic mesh. The synthetic mesh may be made of one or more of synthetic materials. The synthetic material may be a synthetic polymer may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The mesh may be flat, for example, in the form of a sheet.

The synthetic material may be a synthetic suture. The synthetic suture may be made of one or more synthetic materials. The synthetic material may be a synthetic polymer and may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof. The suture may be in an elongated shape, and pass through a material (e.g., tissue) to connect two or more different materials.

According to the repair method of the present invention, the synthetic material may be a synthetic tubular bridging device. The bridging device may be made of one or more synthetic materials. The synthetic material may be a synthetic polymer and may be selected from the group consisting of polylactic acid, polyglycolic acid, poly-4-hydroxybutyrate, polydioxanone, trimethylene carbonate, polycaprolactone, silk, silk fibroin, polypropylene, polyester, polyethylene, polyamide, polyurethane, poly(methyl methacrylate), polytetrafluoroethylene, polydimethylsiloxane, and a combination thereof.

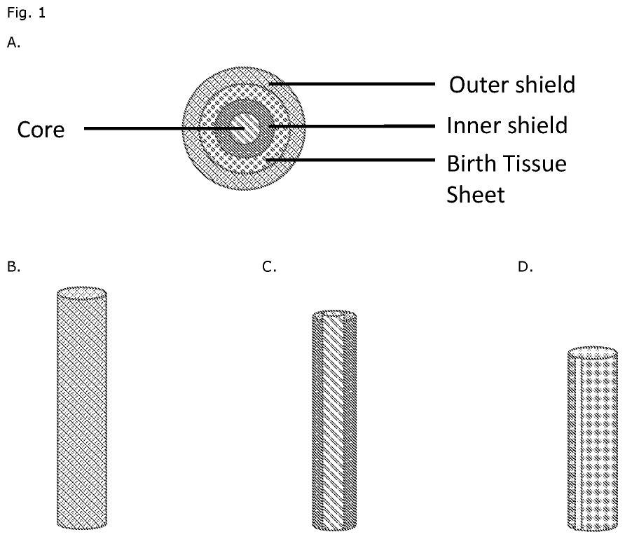

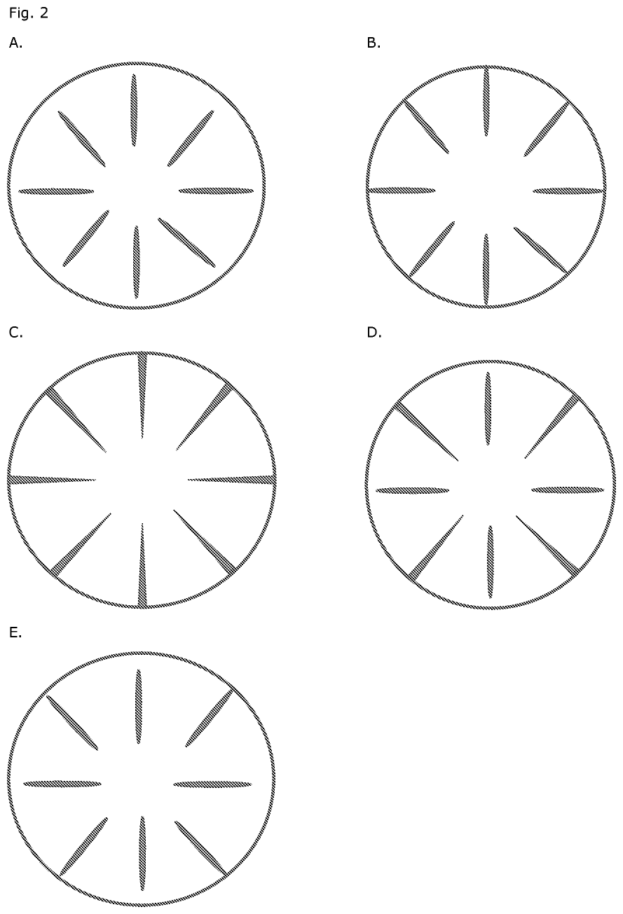

According to the repair method of the present invention, the birth tissue derived implant may be delivered with a delivery device. The delivery device may be in a rod shape, also known as a tubular shape. The delivering device may comprise a core, an inner shield and an outer shield (FIGS. 1A-D). The core may comprise a stapler, glue dispenser, suture device, or combination thereof. The diameter of the tubular shape may be between 5-30 mm, between 5-20 mm, or between 5-15 mm.

The birth tissue derived implant may be located between the inner shield and outer shield. The birth tissue derived implant may comprise a placental membrane having a trophoblast layer facing the outer shield and an amnion layer facing the inner shield. The birth tissue derived implant may comprise an umbilical cord layer having a Wharton's Jelly layer facing the outer shield and an umbilical cord lining facing the inner shield. The outer shield may be removed after the delivery device passes through a cannula or passage device, which connects outside of a human body with a joint, such that the trophoblast layer or Wharton's Jelly layer may be placed on the defective tissue surface by rolling the delivery device until the entire birth tissue is transferred onto the defectives tissue surface. In one embodiment, the repair method may further comprise inserting the device to a surgical space, removing the outer shield from the device, rolling the delivery device on the defective tissue surface, and placing the placental membrane or Wharton's Jelly layer onto the defective tissue surface, and placing the birth tissue derived implant onto the defective tissue surface. As a result, the trophoblast layer or Wharton's Jelly layer may be in contact with the defective tissue surface.

Where the implant birth tissue comprises a placental membrane having a trophoblast layer facing the outer shield and an amnion layer facing the inner shield, the repair method may further comprise: (a) inserting the delivery device to a surgical space where the defective tissue is located; (b) removing the outer shield from the delivery device; and (c) rolling the delivery device after step (b) on the defective tissue surface such that the trophoblast layer would be in contact with the defective tissue surface.

Where the implant birth tissue comprises an umbilical cord having a Wharton's Jelly layer facing the outer shield and an umbilical cord lining facing the inner shield, the repair method may further comprise: (a) inserting the delivery device to a surgical space; (b) removing the outer shield from the delivery device; and (c) rolling the delivery device after step (b) on the defective tissue surface such that the Wharton's Jelly layer would be in contact with the defective tissue surface.

According to the repair method of the present invention, the agent may comprise a first birth tissue elute. The first birth tissue elute may be prepared from a first birth tissue isolated from an amniotic sac, umbilical cord, placental plate, or combination thereof. The first birth tissue elute may be prepared from the amniotic sac, umbilical cord, placental plate, or combination thereof from which the implant birth tissue is isolated. The first birth tissue elute may comprise a growth factor, cytokine, extracellular matrix molecule, protease inhibitor, extracellular vesicle, or combination thereof.

According to the repair method of the present invention, the agent may comprise first birth tissue particulates. The first birth tissue particulates are particulates of a first birth tissue isolated from an amniotic sac, umbilical cord, placental plate, or combination thereof. The first birth tissue particulates may be prepared from the amniotic sac, umbilical cord, placental plate, or combination thereof from which the implant birth tissue is isolated. The first birth tissue particulates may comprise a growth factor, cytokine, extracellular matrix molecule, protease inhibitor, extracellular vesicle, or combination thereof.

According to the repair method of the present invention, the agent may comprise a radioprotectant. The radioprotectant may be endogenous or exogenous to the amniotic sac, umbilical cord, placental plate, or combination thereof from which the implant birth tissue is isolated. The radioprotectant may be a biological factor. The biological factor may be a recombinant biological factor, for example, basic fibroblast growth factor (bFGF). The biological factor may be an antioxidant, also known as free radical scavenger, for example, ascorbic acid, vitamin E, and polysaccharide. The biological factor may be a lubricating factor, for example, hyaluronic acid (HA) or lubricin. The biological factor may be an immunomodulatory and homeostasis factor, for example, interleukin-10 (IL-10) and transforming growth factor beta-3 (TGFβ-3). The biological factor may be an anti-inflammatory factor. The anti-inflammatory factor may be interleukin-1 receptor antagonist (IL-1Ra), hepatocyte growth factor (HGF) or pentraxin 3 (PTX3). The biological factor may be a protease inhibitor. The protease inhibitor may be tissue inhibitor of metalloproteinase 1 (TIMP1), tissue inhibitor of metalloproteinase 2 (TIMP2), tissue inhibitor of metalloproteinase 3 (TIMP3), tissue inhibitor of metalloproteinase 4 (TIMP4), or alpha2 macroglobulin (A2M).

According to the repair method of the present invention, the agent may comprise a first birth tissue elute, first birth tissue particulates, or a combination thereof and a radioprotectant, which may be polysaccharides added to the birth tissue elute before the birth tissue elute is subject to terminal gamma-irradiation for sterilization.

The defective tissue may be a bone tissue (e.g., sub-chondral bone), periosteum, cartilage, soft tissue, spinal cord, dura mater, membrane, or mucosa. Examples of defective soft tissues include tendon, ligament, dermis, skin, vocal cord, nerve, bladder, vagina, urethral, heart, subcutaneous tissue, fascia, breast, muscle, synovium, bursa, ocular membrane, cornea membrane, placental membrane, placenta, and rotator cuff. The defective tissue may be in the musculoskeletal system, digestion system, cardiovascular system, respiratory system, urinary system, reproductive system, nervous system, and/or immune system.

According to the repair method, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer are on opposite surfaces of the first umbilical cord layer. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface. The repair method may further comprise delivering the birth tissue derived implant to a defective tissue surface of a defective tissue in a subject. Where the subject suffers from synovitis, bursitis, or osteoarthritis (OA), and the defective tissue is in a joint having an interior joint cavity and forms a defective interior joint surface, the repair method may further comprise placing the birth tissue derived implant into the joint and suturing the birth tissue derived implant onto the defective interior joint surface so that the Wharton's Jelly layer faces the defective interior joint surface and the umbilical cord lining faces the interior joint cavity. The repair method may further comprise contacting the first Wharton's Jelly layer with the defective tissue. The joint may be a knee, ankle, facet spine, wrist, finger, toe, hip, shoulder or elbow joint. In one embodiment, to prevent the soluble factors from being rinsed away from the birth tissue, and/or to facilitate the insertion step, the birth tissue can be positioned in a non-permeable pouch or delivery container then inserted into the joint and sutured/stapled onto the joint or cartilage surface. After the birth tissue is placed onto the surface, the pouch can be removed.

According to the repair method, the implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, and the first amnion layer and the first trophoblast layer may be on opposite surfaces of the first placental membrane. The repair method may further comprise delivering the birth tissue derived implant to a defective tissue surface of a defective tissue in a subject. Where the subject suffers from synovitis, bursitis, or osteoarthritis (OA), and the defective tissue is in a joint having an interior joint cavity and forms a defective interior joint surface, the repair method may further comprise placing the birth tissue derived implant into the joint and suturing the birth tissue derived implant onto the defective interior joint surface so that the trophoblast layer faces the defective interior joint surface and the amnion layer faces the interior joint cavity. The repair method further comprises contacting the first trophoblast layer with the defective tissue. The joint may be a knee, ankle, facet spine, wrist, finger, toe, hip, shoulder or elbow joint.

The repair method may further comprise delivering the birth tissue derived implant in an open surgery or a minimum invasive surgery. The minimum invasive surgery may an arthroscopic procedure.

Where the defective tissue is a cartilage having a defective cartilage surface, the cartilage repair method may further comprise placing the birth tissue derived implant onto the defective cartilage surface. The birth tissue derived implant may be placed in a surgery. The surgery may be an arthroscopic, a mini-cut, or an open surgery.

According to the method for cartilage repair, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may face the defective tissue surface. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface.

According to the method for cartilage repair, the implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the defective tissue surface.

The method for cartilage repair may further comprise fixing the birth tissue derived implant to the defective cartilage surface by a suture, suture anchor, staple, or glue.

Where the defective tissue is a defective dura that the defect led to an opening between epidural space and spinal cord, the dura repair method may further comprise closing the opening with sutures or grafts and placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the umbilical cord lining may face the epidural space and the Wharton's Jelly layer may face the dura. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the dura and the amnion layer may face the epidural space.

Where the defective tissue is a defect on ligamentum flavum, arachnoid membrane, or other sounding soft tissue after laminectomy, the repair method may further comprise placing the birth tissue derived implant onto the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may face the defective tissue surface and the umbilical cord lining may face the epidural space toward dura, or the Wharton's Jelly layer may face the dura surface and the umbilical cord lining may face the epidural space toward ligamentum flavum, arachnoid membrane, or other sounding soft tissue. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the trophoblast layer may face the defective tissue surface and the amnion layer may face the epidural space toward dura, or the trophoblast layer may face the dura surface and the amnion layer may face the epidural space toward the ligamentum flavum, arachnoid membrane, or other sounding soft tissue.

Where a bone site defect is under the cartilage, the method for cartilage repair may further comprise placing a bone graft into the bone site defect before placing the birth tissue derived implant. The bone graft may comprise viable bone cells and/or osteoprogenitor cells. The bone graft may exclude viable cells. The bone graft may have been freeze-dried, lyopreserved, cryopreserved or devitalized. The bone graft may further comprise a birth tissue elute and/or birth tissue particulates. The bone graft may have an uneven surface. The uneven surface may facilitate attachment of the birth tissue derived implant to an adjacent surface.

Where the defective tissue is a bone, the repair method may further comprise placing the birth tissue derived implant onto the defective tissue surface. The birth tissue derived implant may be placed in a surgery. In one embodiment, the birth tissue derived implant may be wrapped around the defective bone surface. The repair method may further comprise placing the birth tissue derived implant around the defective tissue surface. The implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, the first umbilical cord lining and the first Wharton's Jelly layer are on opposite surfaces of the first umbilical cord layer, and the Wharton's Jelly layer may face the defective bone surface and the umbilical cord lining may face the surrounding soft tissue. The implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, the first amnion layer and the first trophoblast layer may be on opposite surfaces of the first placental membrane, and the amnion layer may face the surrounding soft tissue and the trophoblast layer may face the defective bone surface.

According to the method bone repair, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer are on opposite surfaces of the first umbilical cord layer. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface.

According to the method for bone repair, the implant birth tissue may comprise a first placental membrane isolated from a first amniotic sac. The first placental membrane may comprise a first amnion layer from the first amniotic sac and a first chorion layer from the first amniotic sac. The first chorion layer may comprise a first trophoblast layer from the first amniotic sac. The first amnion layer and the first trophoblast layer may be on opposite surfaces of the first placental membrane.

The method for bone repair may further comprise fixing the birth tissue derived implant to the defective bone surface by a suture, suture anchor, staple, or glue.

Where the defective tissue is a defective nerve tissue having a defective nerve site, for example, a severed nerve segment, the repair method may comprise placing the birth tissue derived implant around the defective nerve site or inserting the birth tissue derived implant in the defective nerve site. The repair method may further comprise delivering the implant birth tissue within a lumen of a tubular bridging device. Where the defective tissue is a defective or severed nerve segment, the repair method may comprise securing the birth tissue at the bridging/suturing sites, and around the luminal surface of the synthetic tubular bridging device with either the trophoblast layer or Wharton's Jelly layer or the amnion layer or umbilical cord lining layer facing the lumen.

According to the method for nerve repair, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer may be on opposite surfaces of the first umbilical cord layer. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface.

According to the method for nerve repair, the implant birth tissue may comprise a first placental membrane isolated from a first amniotic sac, the first placental membrane may comprise a first amnion layer from the first amniotic sac and a first chorion layer from the first amniotic sac, the first chorion layer may comprise a first trophoblast layer from the first amniotic sac, and the first amnion layer and the first trophoblast layer may be on opposite surfaces of the first placental membrane.

According to the repair method, the birth tissue derived implant may be delivered in a non-permeable container to the defective tissue, and the repair method may comprise removing the non-permeable container so that the birth tissue derived implant is contact with the defective tissue.

A method for preparing a birth tissue derived implant is provided. The birth tissue derived implant is prepared for repairing a defective tissue in a subject. The preparation method comprises devitalizing and/or decellularizing an implant birth tissue isolated from an amniotic sac, umbilical cord, placental plate, or combination thereof. The preparation method further comprises adding an agent exogenous to the implant birth tissue to the devitalized and/or decellularized birth tissue. As a result, a birth tissue derived implant is prepared.

According to the preparation method, the agent may comprise a synthetic material. The synthetic material may comprise a synthetic polymer mesh, a synthetic suture, a synthetic tubular bridging device, or a combination thereof.

According to the preparation method, the implant birth tissue may comprise a first umbilical cord layer isolated from a first umbilical cord, the first umbilical cord layer may comprise a first umbilical cord lining from the first umbilical cord and a first Wharton's Jelly layer from the first umbilical cord, and the first umbilical cord lining and the first Wharton's Jelly layer are on opposite surfaces of the first umbilical cord layer. The umbilical cord may be cut open longitudinally, for example, through a blood vessel wall in the umbilical cord, and the blood vessels in the umbilical cord may be substantially removed, for example, by at least about 70%, 80% s, 90%, 95% or 99%. The implant birth tissue may exclude an amnion layer from the first umbilical cord. The first Wharton's Jelly layer may have an uneven surface.

The preparation method may further comprise treating the umbilical cord layer with first birth tissue particulates. The first birth tissue particulates are particulates of a first birth tissue isolated from a first amniotic sac, a first umbilical cord, a first placental plate, or a combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue may not have been treated with an enzyme. The first birth tissue particulates may or may not comprise viable cells. The first birth tissue particulates may have been cryopreserved. The first birth tissue particulates may have been lyophilized or frozen.

The preparation method may further comprise treating the umbilical cord layer with a first birth tissue elute. The first birth tissue elute may be prepared from a first birth tissue isolated from a first amniotic sac, a first umbilical cord, a first placental plate or a combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue elute may be prepared using the methods as described in International Application No. PCT/US2020/028604. To prepare a first birth tissue elute, the first birth tissue particulates may be mixed with a liquid to form a mixture. After the mixture is incubated, a supernatant may be collected from the mixture as a first birth tissue elute. The first birth tissue may not have been treated with an enzyme. The first birth tissue particulates may or may not comprise viable cells. The first birth tissue particulates may have been cryopreserved. The first birth tissue particulates may have been lyophilized or frozen.

According to the preparation method, the implant birth tissue may comprise a first placental membrane isolated from a first amniotic sac. The first placental membrane may comprise a first amnion layer from the first amniotic sac and a first chorion layer from the first amniotic sac. The first chorion layer may comprise a first trophoblast layer from the first amniotic sac. The first amnion layer and the first trophoblast layer may be on opposite surfaces of the first placental membrane.

The preparation method may further comprise treating the placental membrane with a first birth tissue elute. The first birth tissue elute may be prepared from a first birth tissue isolated from a first amniotic sac, a first umbilical cord, a first placental plate or a combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue elute may be prepared using the methods as described in International Application No. PCT/US2020/028604. To prepare a first birth tissue elute, the first birth tissue particulates may be mixed with a liquid to form a mixture. After the mixture is incubated, a supernatant may be collected from the mixture as a first birth tissue elute. The first birth tissue may not have been treated with an enzyme. The first birth tissue particulates may or may not comprise viable cells. The first birth tissue particulates may have been cryopreserved. The first birth tissue particulates may have been lyophilized or frozen.

The preparation method may further comprise treating the placental membrane with first birth tissue particulates. The first birth tissue particulates are particulates of a first birth tissue isolated from a first amniotic sac, a first umbilical cord, a first placental plate, or a combination thereof. The first birth tissue and the implant birth tissue may be isolated from the same amniotic sac, umbilical cord, placental plate, or combination thereof. The first birth tissue and the implant birth tissue may be isolated from different amniotic sacs, umbilical cords, placental plates or combinations thereof. The first birth tissue may not have been treated with an enzyme. The first birth tissue particulates may or may not comprise viable cells. The first birth tissue particulates may have been cryopreserved. The first birth tissue particulates may have been lyophilized or frozen.

The preparation method may further comprise drying, freeze-drying, plasticizing and/or cryopreserving the placental membrane.

The preparation method may further comprise decellularizing the placental membrane, for example, without separating the amnion layer from the chorion layer in the placental membrane as described in International Application No. PCT/US2016/068526. The amnion layer and the chorion layer may remain in contact with each other with a contact surface of, for example, at least about 1, 10, 100, 1,000 or 10,000 mm² during the step of removing cells from a pre-decellularized placental membrane to produce a decellularized placental membrane. The contact surface between the amnion layer and the chorion layer may overlap with at least about 5, 10, 15, 20, 25, 30, 35, 40, 50, 60, 70, 80, 90, 95, 98 or 99% of the surface of the amnion layer facing the chorion layer. The contact surface may overlap with at least about 5, 10, 15, 20, 25, 30, 35, 40, 50, 60, 70, 80, 90, 95, 98 or 99% of the surface of the chorion layer facing the amnion layer.

The cell removal or decellularizing process described herein may be performed in accordance with the methods described in U.S. Pat. Nos. 6,734,018, 6,743,574, 7,338,757, 8,574,826, and 8,563,232, and U.S. Patent Application Publication No. 2014/0065238A1 and 2014/0154663A1, each of which is incorporated by reference herein in its entireties. The decellularizing process may be performed without damage to matrix and/or tissue structure of the placental membrane and may employ sarcosinates and decontaminating agents. In some embodiments, the decellularizing process may or may not include using detergent, endonuclease, and/or protease. The decellularized placental membrane may include collagens, hyaluronins, elastins, mucopolysaccharides and proteoglycans, among other components. In some embodiments, the decellularized placental membrane may comprise more than one type of collagen and/or more than one proteins, other than collagen, collagen type I, and/or collagen type IV.

In some embodiments, the decellularized placental membrane comprises less than about 1000, 500, 100, 80, 50 or 10 ng double-stranded DNA (dsDNA) per mg dry weight of the decellularized placental membrane. In other embodiments, the DNA quantity in the decellularized placental membrane is reduced by about 60, 70, 80, 85, 90, 95, 99% or more; about 60, 70, 80, 85, 90, 95, 99, 100% or less; and/or between about 50 and 100%, between about 70 and 100%, between about 90 and 100%, between about 90 and 95% compared to the DNA quantity in the pre-decellularized placental membrane. The decellularized placental membrane may comprise DNA in an amount that is less than about 50, 40, 30, 20, 10, 5 or 1% of the DNA in the pre-decellularized placental membrane.

The decellularized placental membrane may comprise one or more growth factors and/or cytokines (e.g., platelet-derived growth factor (PDGF), basic fibroblast growth factor (bFGF), epidermal growth factor (EGF), granulocyte-colony stimulating factor (GCSF), placenta growth factor (PIGF), fibroblast growth factor (FGF), transforming growth factor (TGF), macrophage inflammatory protein, interleukins, insulin-like growth factor (IGF), and insulin-like growth factor binding protein (IGFBP)) in an amount that is at least about 1, 5, 10, 15, 20, 25 or 30% greater than the sum of the amount of the one or more growth factors in a decellularized control isolated amniotic membrane and the amount of the one or more growth factors in a decellularized control isolated chorionic membrane having a substantially identical area size. The term “substantially identical area size” as used herein refers to an area size, for example, the size of a section area of a decellularized placental membrane, that is less than about 20, 15, 10, 5 or 1% different from a control area size, for example, the area size of a decellularized isolated amniotic or chorionic membrane. The decellularized control isolated amniotic membrane and the decellularized control isolated chorionic membrane may be prepared from the amniotic sac used to prepare the decellularized placental membrane.

The decellularized placental membrane may comprise one or more growth factors and/or cytokines (e.g., platelet-derived growth factor (PDGF), basic fibroblast growth factor (bFGF), epidermal growth factor (EGF), granulocyte-colony stimulating factor (GCSF), placenta growth factor (PIGF), fibroblast growth factor (FGF), transforming growth factor (TGF), macrophage inflammatory protein, interleukins, insulin-like growth factor (IGF), and insulin-like growth factor binding protein (IGFBP)) in an amount that is at least about 1, 5, 10, 15, 20, 25 or 30% of the amount of the one or more growth factors in the pre-decellularized placental membrane.

According to the preparation method, the implant birth tissue may comprise a placental plate. The implant birth tissue may consist of a placental plate. The placenta plate may be in the form of particulates. The implant birth tissue consisting of a placental plate does not comprise an amniotic sac, an umbilical cord, an amnion layer or a chorion layer.

The preparation method may further comprise treating the placental plate particulate with a first birth tissue elute. The first birth tissue elute may be prepared from a first birth tissue isolated from a first amniotic sac, a first umbilical cord, a first placental plate or a combination thereof. The first birth tissue and the implant birth tissue may be isolated from the same amniotic sac, umbilical cord, placental plate, or combination thereof. The first birth tissue elute and the implant birth tissue may be prepared from the same tissue isolated from an amniotic sac, umbilical cord, placental plate, or combination thereof, where a soluble fraction of the isolated tissue is removed from the isolated tissue to prepare the first birth tissue elute while the resulting isolated tissue is used to prepare the implant birth tissue, with or without further processing, for example, decellularization. The first birth tissue elute may be added to the implant birth tissue. The first birth tissue and the implant birth tissue may be isolated from different amniotic sacs, umbilical cords, placental plates or combinations thereof. The first birth tissue elute may be prepared using the methods as described in International Application No. PCT/US2020/028604. To prepare a first birth tissue elute, the first birth tissue particulates may be mixed with a liquid to form a mixture. After the mixture is incubated, a supernatant may be collected from the mixture as a first birth tissue elute. The first birth tissue may not have been treated with an enzyme. The first birth tissue particulates may or may not comprise viable cells. The first birth tissue particulates may have been cryopreserved. The first birth tissue particulates may have been lyophilized or frozen.

For each preparation method of the present invention, a birth tissue derived implant prepared according to the method is provided.

As used herein, the term “about” modifying, for example, the dimensions, volumes, quantity of an ingredient in a composition, concentrations, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8, 7, 6, 5, 4, 3, 2, 1 percent or less of the stated reference value.

### Example 1. Preparation of Birth Tissues from Umbilical Cord, Placental Membrane, and Amniotic Membrane Derived from Native Placental Plate

Human full term placental plate along with an umbilical cord and an amniotic sac with research consent were obtained after caesarean section and transferred to a processing facility under sterile condition. The umbilical cord, amniotic sac or placental plate may be processed and used as a birth tissue in a birth tissue derived implant according to the present invention.

Umbilical cord:

The umbilical cord attached to the placental plate was cut at the cord-placenta junction and rinsed with ice-cold saline. Umbilical cord was further cut into segments (5-6 cm long) in the saline and loosely bond blood clots along the umbilical cords were removed. Umbilical cord segments were transferred to a plastic petri dish for dissection. A longitudinal cut was made by a scalpel along the length of the umbilical cord segment and to the depth of the vein to cut open and expose the luminal side of the umbilical cord vein. The endothelial cells and wall of the umbilical vein were substantially scraped off with the scalpel. The arteries were then dissected out from the surrounding tissues with forceps and scissors. The processed umbilical cord layer were cut into small pieces (˜0.5 cm) and transferred to a container and stored at −80° C. until further process.

Amniotic sac:

The amniotic sac attached to the placental plate was cut and rinsed in ice-cold saline to remove blood clots. The processed amniotic sac was further decellularized and lyophilized to make lyophilized decellularized placental membrane (Decell PM).

Amniotic membrane from placental plate:

An amniotic membrane was gently peeled off from the placental plate and rinsed in ice-cold saline to remove blood clots. The processed amniotic membrane was lyophilized to make a lyophilized amniotic membrane (AM).

### Example 2. Preparation of Umbilical Cord Particulates

The frozen umbilical cords in small pieces (˜0.5 cm) made as described in Example 1 were used for micronization. A cryomill (Retsch) or a grinder was used to micronize the small frozen, umbilical cords pieces. The frozen umbilical cord pieces were transferred to a grinding jar with a grinding ball, and then the jar was sealed. The grinding jar was pre-cooled by liquid nitrogen prior to the grinding process. The frozen umbilical cord pieces were pulverized for 15 mins at 30 Hz with the grinding jar being continually cooled with liquid nitrogen. As a result, the umbilical cords were micronized into umbilical cord particulates.

### Example 3. Preparation of an Umbilical Cord Elute

A DMEM medium or appropriate medium with 1% antibiotics was added to the umbilical cord particulates made from Example 2 at 2 mL medium per gram of the umbilical cord particulates in 50 mL conical tubes to form a medium-umbilical cord particulate suspension, which was mixed well and placed on a rocker with agitation for 24-40 hours at 4° C.

At the end of the incubation, the medium-umbilical cord suspension was centrifuged at 3,000 rpm for 25 minutes. The supernatant was collected.

The collected supernatant, also called umbilical cord elute, umbilical cord conditioned medium, micronized umbilical cord conditioned medium or micronized umbilical cord elute was aliquoted and stored at −80° C. or lyophilized and stored at ambient temperature. The umbilical cord conditioned medium may be used as an injectable formula, alone or in combination with other tissues.

The umbilical cord particulates in the pellets following the centrifugation were also collected, aliquoted and stored at −80° C. or lyophilized.

### Example 4. Preparation of Freeze Dried, Enhanced Decellularized Placental Membrane or Amniotic Membrane

The freeze dried, decellularized placental membrane (Decell PM) and the freeze dried amniotic membrane (AM) derived from placental plate prepared as described in Example 1 were submerged in the umbilical cord elute prepared as described in Example 3 with gentle agitation at 4° C. overnight. Then the elute soaked decell PM and AM were further lyophilized to make lyophilized enhanced Decell PM and lyophilized enhanced AM, respectively.

### Example 5. Biological Factor Quantification of Lyophilized Amniotic Membrane and Lyophilized Enhanced Amniotic Membrane

The lyophilized AM and lyophilized enhanced AM prepared as described in Examples 1 and 4, respectively, were digested by collagenase, and the tissue digestions were used for quantification of hyaluronic acid (HA), alpha-2 macroglobulin (A2M) and tissue inhibitor of metalloproteinase 1 (TIMP1) using Luminex kits (R&D systems). It was found that the lyophilized enhanced AM contained more HA, A2M and TIMP1 than the lyophilized AM. The results are summarized in Table 1.

TABLE 1
Biological factors

| (Biological factor/mm²} | AM | Enhanced AM |
|---|---|---|
| HA (ng/mm²) | 146.1 | 278.7 |
| A2M (ng/mm²) | 1.5 | 3.0 |
| TIMP1 (pg/mm²) | 23.7 | 51.6 |

### Example 6. Anti-Inflammatory Effect of Lyophilized, Decellularized Placenta Membrane and Lyophilized, Enhanced, Decellularized Placental Membrane

The anti-inflammatory effects of the lyophilized decellularized PM (Decell PM) and lyophilized, enhanced Decell PM on inhibition of TNF-alpha secretion by THP1 cells after lipopolysaccharide stimulation, a human macrophage cell line, were studied. The lyophilized Decell PM and the lyophilized, enhanced Decell PM were prepared as described in Examples 1 and 4, respectively. Human TNF-alpha Luminex kits (R&D systems) were used to determine the secretion levels of TNF-alpha from the THP1 cells after being treated with extractions from either the lyophilized Decell PM or the lyophilized, enhanced, Decell PM.

Day 1: THP1 cells were seeded in culture plates having an appropriate culture medium with 200 ng/mL phorbol myristate acetate (PMA) and incubated.

Day 2: The cells were washed with a fresh culture medium.

Day 3: The cells were then stimulated with 1 ug/mL lipopolysaccharide (LPS, Sigma) for 24 hrs. Extractions of the lyophilized Decell PM and the lyophilized, enhanced Decell PM were also prepared by incubating both Decell PM types at 200 mm²/mL in culture medium with gentle agitation at 4° C.

Day 4: The medium in the culture plate was replaced, and cells were treated with the Decell PM extractions and 1 ug/mL LPS. Twenty-four hours following the last LPS stimulation, supernatants from the culture plates were collected and frozen in −80° C. for storage until the TNF-alpha Luminex assay. As shown in FIG. 4, the extraction from the lyophilized, enhanced Decell PM showed greater TNF-α inhibitory effect than that from the lyophilized Decell PM (55.2% vs. 36.9%).

### Example 7. MMP1 Inhibition Effect of Lyophilized Amniotic Membrane and Lyophilized, Enhanced, Amniotic Membrane

The MMP1 inhibitory effects of lyophilized amniotic membrane (AM) and lyophilized, enhanced AM were studied. The lyophilized AM and the lyophilized, enhanced AM were prepared as described in Examples 1 and 4. Extractions of AM and enhanced AM were prepared by incubating both AM types at 50 mm²/mL in an appropriate buffer with agitation at 4° C. The extraction was the supernatant obtained after centrifugation of the tissue suspension. The extractions of both AM types were incubated with MMP1 enzymes at 50 ng/mL for 10 mins at 37° C. The supernatants were collected, added to a 96-well plate and mixed with an MMP1 enzyme substrate. Dynamic absorbance readings were performed and a higher O.D. value corresponds to a higher MMP1 enzyme activity. The results showed that the extraction from the lyophilized, enhanced AM led to higher MMP1 inhibitory effect when compared to that from the lyophilized AM (7.3% vs. 0.5%).

### Example 8. Preparation of Decellularized Intact Placenta Plate and Decellularized Placenta Plate Particulates

Protocols for preparing decellularized intact placenta plate and decellularized placenta plate particulates are provided.

### Intact Placenta Plate, Decellularized

Following recovery, the placenta plate was separated from the placenta membrane and umbilical cord by sharp dissection. Blood clots were removed, the plate was rinsed in water, and frozen until future use. The tissue was thawed, rinsed with water, and secured to a polymer mesh. Optionally, the plate's thickness may be reduced by removing some of the chorionic villous tissue by sharp resection. The tissue was sequentially treated with red blood cell lysis solution, rinsed in water, treated with a decellularization solution containing sodium lauroyl sarcosine (NLS), and rinsed in saline. Optionally, the material may be compressed prior to lyophilization. The material was frozen and lyophilized to yield a dry intact placenta plate implant. The implant was cut to size and sterilized prior to use.

### Placenta Plate Particulates, Decellularized

Method 1. Dry sheets of intact placenta plate were comminuted to particulates through a dry milling process. Optionally, particles may be screened for particle fractions of desired size. The particulates were sterilized prior to use.

Method 2. Following recovery, the placenta plate was separated from the placenta membrane and umbilical cord by sharp dissection. Blood clots were removed, the plate was rinsed in water, cut into chunks, and frozen until future use. The tissue was thawed, rinsed in water, and ground into small, millimeter-sized particles. The tissue was sequentially treated with red blood cell lysis solution, rinsed in water, and treated with a decellularization solution containing sodium lauroyl sarcosine (NLS). The decellularized material was rinsed in saline and frozen until further use. Optionally, the material's particle size may be further reduced using a cryogenic milling process. Preparation of placenta plate particulates included: The frozen particulates were dried by lyophilization to yield a dry particulate. Optionally, particles may be screened for particle fractions of desired size. Preparation of reconstituted particulate sheets and shapes (i.e., cylinder): (a) The material was blended with ice to yield a wet paste and cast into a sheet or any desired shape under compression (optional) prior to lyophilization; (b) Lyophilization yielded a dry implant composed of reconstituted placenta plate particulates; (c) The implant was cut to size; and (d) The powder or implant was sterilized prior to use.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A birth tissue derived implant, comprising an implant birth tissue and an agent exogenous to the implant birth tissue, wherein the implant birth tissue comprises a first placental membrane isolated from a first amniotic sac, wherein the first placental membrane comprises a first amnion layer from the first amniotic sac and a first chorion layer from the first amniotic sac, wherein the first chorion layer comprises a first trophoblast layer from the first amniotic sac, and wherein the first amnion layer and the first trophoblast layer are on opposite surfaces of the first placental membrane, and wherein the agent comprises a synthetic material.

2. (canceled)

3. The birth tissue derived implant of claim 1, wherein the agent comprises a first birth tissue elute, first birth tissue particulates, a radioprotectant, a biological factor, an immunomodulatory factor, a homeostasis factor, an antiinflammatory factor, a protease inhibitor, an antioxidant, or a combination thereof.

4. The birth tissue derived implant of claim 3, wherein the first birth tissue elute comprises a growth factor, a cytokine, an extracellular matrix molecule, an extracellular vesicle, a protease inhibitor, or a combination thereof.

5. The birth tissue derived implant of claim 1, wherein the implant birth tissue comprises a second birth tissue elute, second birth tissue particulates, or a combination thereof.

6. The birth tissue derived implant of claim 1, wherein the birth tissue derived implant further comprises a third birth tissue elute, third birth tissue particulates, or a combination thereof.

7. The birth tissue derived implant of claim 1, wherein the implant birth tissue is fenestrated, meshed, multi-layered, or crosslinked.

8-11. (canceled)

12. The birth tissue derived implant of claim 1, wherein the implant birth tissue comprises cells exogenous to the implant birth tissue.

13. A birth tissue derived implant, comprising an implant birth tissue and an agent exogenous to the implant birth tissue, wherein the implant birth tissue comprises a first umbilical cord layer isolated from a first umbilical cord, wherein the first umbilical cord layer comprises a first umbilical cord lining from the first umbilical cord and a first Wharton's Jelly layer from the first umbilical cord, and wherein the first umbilical cord lining and the first Wharton's Jelly layer are on opposite surfaces of the first umbilical cord layer.

14. The birth tissue derived implant of claim 13, wherein the implant birth tissue further comprises a second umbilical cord layer isolated from the first umbilical cord or a second umbilical cord, wherein the second umbilical cord layer comprises a second umbilical cord lining from the first umbilical cord or the second umbilical cord and a second Wharton's Jelly layer from the first umbilical cord or the second umbilical cord, wherein the second umbilical cord lining and the second Wharton's Jelly layer are on opposite surfaces of the second umbilical cord layer.

15. The birth tissue derived implant of claim 14, wherein the first umbilical cord lining is in contact with the second umbilical cord lining.

16. The birth tissue derived implant of claim 14, wherein the first Wharton's Jelly layer is in contact with the second Wharton's Jelly layer.

17-19. (canceled)

20. The birth tissue derived implant of claim 1, wherein the implant birth tissue further comprises a second placental membrane isolated from the first amniotic sac or a second amniotic sac, wherein the second placental membrane comprises a second amnion layer from the first amniotic sac or the second amniotic sac and a second chorion layer from the first amniotic sac or the second amniotic sac, wherein the second chorion layer comprises a second trophoblast layer from the first amniotic sac or the second amniotic sac, and wherein the second amnion layer and the second trophoblast layer are on opposite surfaces of the second placental membrane.

21. The birth tissue derived implant of claim 1, wherein the first amnion layer is in contact with the second amnion layer or the second trophoblast layer.

22. The birth tissue derived implant of claim 1, wherein the implant birth tissue further comprises a second placental membrane isolated from a second amniotic sac, wherein the second placental membrane comprises a second chorion layer from the second amniotic sac, wherein the second chorion layer comprises a second cellular layer from the second amniotic sac and a second trophoblast layer from the second amniotic sac, and wherein the second cellular layer and the second trophoblast layer are on opposite surfaces of the second placental membrane.

23. (canceled)

24. A birth tissue derived implant, comprising an implant birth tissue and an agent exogenous to the implant birth tissue, wherein the implant birth tissue consists of a placental plate.

25. The birth tissue derived implant of claim 24, wherein the placental plate is in the form of particulates.

26. A method for repairing a defective tissue in a subject, comprising:
(a) delivering the birth tissue derived implant of claim 1 to a defective tissue surface of a defective tissue in a subject; and
(b) placing the birth tissue derived implant in contact with the defective tissue surface.

27-28. (canceled)

29. The method of claim 26, wherein the birth tissue derived implant is located between the inner shield and the outer shield, and wherein the first trophoblast layer faces the outer shield and the first amnion layer faces the inner shield, said method further comprising:
(a) inserting the delivery device to a surgical space where the defective tissue is located;
(b) removing the outer shield from the delivery device; and
(c) rolling the delivery device after step (b) on the defective tissue surface, whereby the first trophoblast layer is in contact with the defective tissue surface.

30-31. (canceled)

32. The method of claim 26, wherein the defective tissue is a defective cartilage, said method further comprising placing the birth tissue derived implant onto the defective tissue surface.

33-41. (canceled)

42. A method for repairing a defective tissue in a subject, comprising:
(a) delivering the birth tissue derived implant of claim 13 to a defective tissue surface of a defective tissue in a subject, wherein the subject suffers from synovitis, bursitis, or osteoarthritis (OA), and the defective tissue is in a joint having an interior joint cavity and forms a defective interior joint surface;
(b) placing the birth tissue derived implant into the joint; and
(c) suturing the birth tissue derived implant onto the defective interior joint surface, wherein the first Wharton's Jelly layer faces the defective interior joint surface and the first umbilical cord lining faces the interior cavity.

43. (canceled)

44. A method for repairing a defective tissue in a subject, comprising:
(a) delivering the birth tissue derived implant of claim 1 to a defective tissue surface of a defective tissue in a subject, wherein the subject suffers from synovitis, bursitis, or osteoarthritis (OA), and the defective tissue is in a joint having an interior joint cavity and forms a defective interior joint surface;
(b) placing the birth tissue derived implant into the joint; and
(c) suturing the birth tissue derived implant onto the defective interior joint surface, whereby the first trophoblast layer faces the defective interior joint surface and the first amnion layer faces towards the interior cavity.

45-48. (canceled)

49. The method of claim 44, wherein the birth tissue derived implant is delivered in a non-permeable container to the defective tissue, further comprising removing the non-permeable container, whereby the birth tissue derived implant is in contact with the defective tissue.

50-59. (canceled)
